(19) United States
(12) Patent Application Publication
(10) Pub. No.: US 2003/0032008 A1
(43) Pub. Date: Feb. 13, 2003

(54) DIagnostic reagents and methods for antibody against hepatitis b surface antigen and for antibody against hepatitis b virus

(76) Inventors: Kook-Jin Lim, Koyang-si (KR); Jae-Hoon Oh, Daejeon-si (KR); Mi-Jin Shon, Daejeon-si (KR); Seung-Bum Yoo, Daejeon-si (KR); Sang-Ik Lee, Daejeon-si (KR)

Correspondence Address: JACOBSON HOLMAN PLLC, 400 SEVENTH STREET N.W., SUITE 600, WASHINGTON, DC, 20004, US

(21) Appl. No.: 10/181,430

(22) PCT Filed: Jan. 31, 2001
(86) PCT No.: PCT/KR01/00127

(30) Foreign Application Priority Data
Feb. 1, 2000 (KR) 2000-4785

(51) Int. Cl. C12Q 1/70; C12N 1/18; C12P 21/02; C12N 5/06

(52) U.S. Cl. 435/5; 435/69.3; 435/254.2; 435/325

(57) ABSTRACT

The present invention relates to diagnostic reagents for antibody against hepatitis B surface antigen and for antibody against hepatitis B virus using a sandwich immunoassay as a quantitative or qualitative analysis method for antibody against hepatitis B surface antigen and for antibody against hepatitis B virus, characterized in that each diagnostic reagent comprises a pair of hepatitis B surface antigens which are different each other and composed of primary antigen immobilized on a solid support and secondary antigen conjugated to a marker. Also, the present invention relates to preparation methods thereof. Further, the present invention relates to diagnostic methods for antibody against hepatitis B surface antigen and for antibody against hepatitis B virus. In accordance with the present invention, there are provided diagnostic reagents and methods with markedly enhanced sensitivity and specificity in comparison to diagnostic reagents and methods in which single kind of hepatitis B virus surface antigens are used.

### TECHNICAL FIELD

The present invention relates to diagnostic reagents for antibody against hepatitis B surface antigen and for antibody against hepatitis B virus, to preparation methods thereof, and to diagnostic methods for antibody against hepatitis B surface antigen and for antibody against hepatitis B virus.

### BACKGROUND ART

Liver, one of the major organs to maintain life, can be damaged by many factors such as alcohol, drugs, etc. One of the most fatal factors to damage the liver is virus infection. Hitherto, five different types of hepatitis viruses, A, B, C, D and E, have been identified as hepatitis virus to damage the liver. Among them, the possibility that damages in hepatocytes caused by hepatitis B virus (HBV) develop into chronic hepatitis and liver cirrhosis by continuous viral multiplication has been reported to be high. In Korea, the incidence rate of hepatitis B is relatively higher than developed countries, and mothers traditionally have much contact with their babies. Accordingly, in Korea, the probability that newborn-babies become carriers of HBV if their mothers are carriers of HBV reaches to 90%. Currently, the carriers of HBV in Korea is 8 to 12% of the total population (Kim, H. S., Yang, D. O., Shin, D. H., Kim, H. W., Kim, S. J. : Follow-up study-of immunity of hepatitis B vaccine for 5 years. Journal of Korean Internal Medicine 41(2): 164˜171, 1991; Kook, Y. H., Park, J. K.: Seroconversion rate by inoculation of hepatitis vaccine. Infection 17(2): 155˜162, 1985).

After discovery of Australia antigen which is a specific hepatitis antigen, hepatitis B virus surface antigen (HBsAg) and the whole HBV particle were discovered by Dane in 1970 in the serum of patients suffering from hepatitis (Seo, J. H., Park, B. C.: Hepatitis virus (1), Life Science 2(3): 163˜174, 1992). In the HBV gene, existence of 6 open reading frames has been discovered to date. Among the proteins expressed by these, 24 kd SHBs (S site), 31 kd MHBs (Pre S₂+S) and 38 kd LHBs (Pre S₁+Pre S₂+S) form the viral surfaces of HBV. HBsAg seems to have p-24 and glycosylated p-30 having different molecular weights each other when identified by electrophoresis from plasma. In natural conditions, HBsAg does not exist as a single protein, but exists as a complex protein containing carbohydrates and lipids. HBsAg exists as a big particle with a molecular weight of about 3,000,000 and a diameter of about 20 nm. When infected by hepatitis B virus, antibodies against HBcAg (hepatitis B core antigen), HBsAg and HBeAg (hepatitis B e antigen) are formed. Among them, only antibody against HBsAg has been identified to have a protective immunity against re-infection of HBV.

Therefore, hitherto, the development of preventive vaccine against hepatitis B virus has aimed to produce antibody against HBsAg by means of administration of the vaccine. Whether one has acquired immunity against hepatitis B or not is determined depending on the existence of antibody against HBsAg. In general, one is considered to have acquired immunity against HBV when his/her antibody concentration against HBsAg is higher than 10 mlU/ml Re-inoculation of vaccine is recommended if the antibody level is lower than 10 mlU/ml.

Diagnostic reagents for detecting antibody against HBsAg have been produced by many companies. The reaction principle makes use of a sandwich method where plasma or serum is added to react with HBsAg immobilized on the solid support(e.g., a plate, beads or the like) so that antibody against HBsAg binds to the surface antigen, and then secondary HBsAg which is labeled with enzyme or isotope or by similar techniques binds to the antibody. Depending on the method of labeling, assay methods used in diagnostic reagents are divided into several categories, Enzyme-linked immunosorbent assay (ELISA), radioimmunoassay (RIA) and the like. Most of diagnostic reagents for detecting antibody against HBsAg which have been currently used utilize HBsAg purified from plasma. HBsAg purified from plasma, however, is not a pure surface antigen protein, but a complex containing carbohydrates and lipids. Therefore, false-positive signal can be detected by not the antibody against HBsAg protein but antibodies against or materials with affinity for components other than protein of HBsAg. Although labeled secondary antigens of low concentrations are used to lower such false-positive signals, in this case, the sensitivity of diagnostic reagent becomes lowered. As an attempt to overcome the low sensitivity of these methods, there was a diagnostic kit (Abbot, USA) using a technique where biotin is covalently bonded to secondary HBsAg and anti-biotin antibody labeled with horseradish peroxidase is used.

### DISCLOSURE OF INVENTION

An object of the present invention is to provide diagnostic reagents for antibody against hepatitis B surface antigen and for antibody against hepatitis B virus with enhanced sensitivity and specificity.

Another object of the present invention is to provide methods of preparing diagnostic reagents for antibody against hepatitis B surface antigen and for antibody against hepatitis B virus.

Another object of the present invention is to provide diagnostic methods for antibody against hepatitis B surface antigen and for antibody against hepatitis B virus with enhanced sensitivity and specificity.

The present invention provides a diagnostic reagent for antibody against hepatitis B surface antigen using a sandwich immunoassay as a quantitative or qualitative analysis method for antibody against hepatitis B surface antigen, characterized in that the diagnostic reagent comprises a pair of hepatitis B surface antigens which are different each other and composed of primary antigen immobilized on a solid support and secondary antigen conjugated to a marker. Further, the present invention provides a diagnostic reagent for antibody against hepatitis B virus using a sandwich immunoassay as a quantitative or qualitative analysis method for antibody against hepatitis B virus, characterized in that the diagnostic reagent comprises a pair of hepatitis B surface antigens which are different each other and composed of primary antigen immobilized on a solid support and secondary antigen conjugated to a marker.

Also, the present invention provides a method of preparing a diagnostic reagent for antibody against hepatitis B surface antigen using a sandwich immunoassay as a quantitative or qualitative analysis method for antibody against hepatitis B surface antigen, characterized in that the method comprises a step of including a pair of hepatitis B surface antigens which are different each other and composed of primary antigen immobilized on a solid support and secondary antigen conjugated to a marker in the diagnostic reagent. Further, the present invention provides a method of preparing a diagnostic reagent for antibody against hepatitis B virus using a sandwich immunoassay as a quantitative or qualitative analysis method for antibody against hepatitis B virus, characterized in that the method comprises a step of including a pair of hepatitis B surface antigens which are different each other and composed of-primary antigen immobilized on a solid support and secondary antigen conjugated to a marker in the diagnostic reagent.

Also, the present invention provides a diagnostic method for antibody against hepatitis B surface antigen using a sandwich immunoassay as a quantitative or qualitative analysis method for antibody against hepatitis B surface antigen, characterized in that the diagnostic method utilizes a pair of hepatitis B surface antigens which are different each other and composed of primary antigen immobilized on a solid support and secondary antigen conjugated to a marker. Further, the present invention provides a diagnostic method for antibody against hepatitis B virus using a sandwich immunoassay as a quantitative or qualitative analysis method for antibody against hepatitis B virus, characterized in that the diagnostic method utilizes a pair of hepatitis B surface antigens which are different each other and composed of primary antigen immobilized on a solid support and secondary antigen conjugated to a marker.

The diagnostic reagents of the present invention comprise two kinds of HBsAgs which are different each other, for example, in terms of origins(e.g., HBsAg obtained from plasma and recombinant HBsAg) or purification methods as a pair of primary and secondary antigens.

Conventional diagnostic reagent for antibody against hepatitis B utilizes the method of detecting the antibody by using hepatitis B virus surface antigen (HBsAg). The HBsAg used in this case is HBsAg derived from human plasma or recombinant HBsAg (Ostrow, D. H. et al, Quantitation of hepatitis B surface antibody by an automated microparticle enzyme immunoassay, Journal of Virological Methods, 32(1991) 265˜276). However, HBsAg is a membrane protein. The pure protein cannot retain its natural conformation by itself Therefore, HBsAg contains a large amount of lipids and carbohydrates. Furthermore, since HBsAg has a particle structure, it is nearly impossible to isolate HBsAg as a pure protein. In other words, hepatitis B surface antigen used in diagnostic reagents for antibody against hepatitis B inevitably contains a large amount of impurities. The present invention can reduce nonspecific reactions by such impurities by means of using a combination of two kinds of HBsAgs which are different each other, for example, in terms of origins or purification methods.

In accordance with the present invention, there are provided diagnostic reagents for antibody against hepatitis B surface antigen and for antibody against hepatitis B virus with enhanced sensitivity and specificity, and preparation methods thereof. Also, in accordance with the present invention, there are provided diagnostic methods for antibody against hepatitis B surface antigen and for antibody against hepatitis B virus with enhanced sensitivity and specificity. These and other features, aspects, and advantages of the present invention will become better understood with reference to the following description and appended claims.

### BEST MODE FOR CARRYING OUT THE INVENTION

The present invention provides diagnostic reagents for antibody against hepatitis B surface antigen and for antibody against hepatitis B virus, preparation methods thereof, and diagnostic methods for antibody against hepatitis B surface antigen and for antibody against hepatitis B virus.

More particularly, the present invention provides a diagnostic reagent for antibody against hepatitis B surface antigen using a sandwich immunoassay as a quantitative or qualitative analysis method for antibody against hepatitis B surface antigen, characterized in that the diagnostic reagent comprises a pair of hepatitis B surface antigens which are different each other and composed of primary antigen immobilized on a solid support and secondary antigen conjugated to a marker. Further, the present invention provides a diagnostic reagent for antibody against hepatitis B virus using a sandwich immunoassay. as a quantitative or qualitative analysis method for antibody against hepatitis B virus, characterized in that the diagnostic reagent comprises a pair of hepatitis B surface antigens which are different each other and composed of primary antigen immobilized on a solid support and secondary antigen conjugated to a marker.

Preferably, the diagnostic reagents of the present invention may comprise surface antigen derived from plasma and recombinant surface antigen as the pair of hepatitis B surface antigens. More preferably, in the diagnostic reagents of the present invention, the recombinant surface antigen may comprise one derived from yeast or one derived from animal cells. Also, the diagnostic reagents of the present invention may comprise two surface antigens which are different each other by different purification methods as the pair of hepatitis B surface antigens. In one embodiment of the diagnostic reagents of the present invention, the primary antigen immobilized on the support may be surface antigen derived from plasma, and the secondary antigen conjugated to the marker may be recombinant surface antigen. In another embodiment of the diagnostic reagents of the present invention, the primary antigen immobilized on the support may be recombinant surface antigen, and the secondary antigen conjugated to the marker may be surface antigen derived from plasma. In the diagnostic reagents of the present invention, the marker may include an enzyme, a radioactive substance, a micro-particle, a dye or the like.

Also, the present invention provides a method of preparing a diagnostic reagent for antibody against hepatitis B surface antigen using a sandwich immunoassay as a quantitative or qualitative analysis method for antibody against hepatitis B surface antigen, characterized in that the method comprises a step of including a pair of hepatitis B surface antigens which are different each other and composed of primary antigen immobilized on a solid support and secondary antigen conjugated to a marker in the diagnostic reagent. Further, the present invention provides a method of preparing a diagnostic reagent for antibody against hepatitis B virus using a sandwich immunoassay as a quantitative or qualitative analysis method for antibody against hepatitis B virus, characterized in that the method comprises a step of including a pair of hepatitis B surface antigens which are different each other and composed of primary antigen immobilized on a solid support and secondary antigen conjugated to a marker in the diagnostic reagent.

Also, the present invention provides a diagnostic method for antibody against hepatitis B surface antigen using a sandwich immunoassay as a quantitative or qualitative analysis method for antibody against hepatitis B surface antigen, characterized in that the diagnostic method utilizes a pair of hepatitis B surface antigens which are different each other and composed of primary antigen immobilized on a solid support and secondary antigen conjugated to a marker. Further, the present invention provides a diagnostic method for antibody against hepatitis B virus using a sandwich immunoassay as a quantitative or qualitative analysis method for antibody against hepatitis B virus, characterized in that the diagnostic method utilizes a pair of hepatitis B surface antigens which are different each other and composed of primary antigen immobilized on a solid support and secondary antigen conjugated to a marker.

The present invention will hereinafter be described in more detail. Hepatitis B virus surface antigen(primary antigen), was immobilized on a solid support such as micro-well plate, membrane or micro-particle and used in order to bind to antibody which is specific to HBsAg. Then, to detect the antibody bound to primary antigen immobilized on the support, another surface antigen(secondary antigen) conjugated to a marker was used. The secondary antigen used was different from primary antigen, for example, in terms of origin or purification method. For example, HBsAg derived from plasma and recombinant HBsAg can be used as primary and secondary antigens, respectively. Also, recombinant HBsAg and HBsAg derived from plasma can be used as primary and secondary antigens, respectively. In these cases, the recombinant HBsAg comprises one derived from yeast, one derived from animal cells or the like. Further, HBsAgs which are purified by different purification methods can be used as primary and secondary antigens, respectively. The concentration of hepatitis B antibody could be determined by using the above marker. The marker conjugated to the secondary antigen, includes radioactive substances such as radioisotopes, enzymes such as alkaline phosphatase or horse radish peroxidase, fluorescent materials or dyes such as micro-particle, colloidal gold, or the like. Using the above method, diagnostic reagents of the present invention can detect hepatitis B antibody with enhanced sensitivity and specificity.

The previously described versions of the present invention have many advantages, including providing diagnostic reagents for antibody against hepatitis B surface antigen and for antibody against hepatitis B virus with enhanced sensitivity and specificity, providing preparation methods thereof, and providing diagnostic methods for antibody against hepatitis B surface antigen and for antibody against hepatitis B virus with enhanced sensitivity and specificity.

The present invention will be described in more detail hereinafter in connection with the following examples, which should be considered as being exemplary only and not limiting the present invention.

### EXAMPLE 1

### Purification of Hepatitis B Virus Surface Antigen from Plasma.

In an ice bath, 59 ml of saturated ammonium sulfate was slowly added to 140 ml of hepatitis B surface antigen positive blood and stirred for 1 hour This solution was centrifuged for 20 minutes at 12,000 rpm in a centrifuge, and 200 ml of supernatant thereof was transferred to a 500-ml glass beaker on an ice bath. To the supernatant, 80 ml of saturated ammonium sulfate was slowly added and stirred for 2 hours. After removing the supernatant, the remainder was dissolved by adding 30 ml of phosphate buffered saline (PBS). This solution was passed through a column packed with 2 L of Sepharose CL-4B gel (Pharmacia, Sweden) equilibrated with PBS. Thereafter, the eluted solution was collected by 40 ml fractions. Antigen titer of each fraction eluted from the column was determined by using LG HBsAg-ELISA (LG household & health Care, Korea) to collect the fractions containing the antigen. To the collected solution, cesium chloride(CsCl) was added to a final concentration of 25% and dissolved, and then it was ultra-centrifuged for 48 hours at 45,000 rpm. After the portions containing the antigens were collected, they were dialyzed three times against 1 L of PBS and stored in a freezer.

### EXAMPLE 2

### Purification of Hepatitis B Virus Surface Antigen from Recombinant Yeast

After 70 g of recombinant yeast, Saccharomyces Cerevisiae pYLBC GAP-UB-HBs/AB110 (Deposit No. KCTC 8722P in Korean Collection for Type Cultures of Korea Research Institute of Bioscience and Biotechnology deposited on Jan. 5, 1996; refer to Korean Patent Number 177307) expressing hepatitis B virus antigen was suspended in cell lysis solution (0.5M NaCl, 10 mM EDTA, 0.1M phosphate buffer, 0.5% Triton X-100, pH 7.0), yeast cells were lysed with glass beads by using Bead Beator (Biospec Product, USA) 5N hydrochloric acid(HCl) was added to this solution to adjust the pH to 3.8, and then clear supernatant of the solution was obtained by centrifugation After the supernatant was titrated to pH 7.0, silica powder (Aerosil-380) was added to the supernatant solution and then the solution was stirred for 12 hours in a cold room to immobilize the surface antigen to the silica. The silica to which the surface antigen was immobilized, was harvested by centrifugation and washed twice with PBS to remove impurities that were not adsorbed. Hepatitis B surface antigen was desorbed from silica by stirring for 3 hours in 50 mM carbonate buffer solution, pH 9.5, and then the solution was centrifuged to obtain the supernatant. Thereafter, through DEAE-chromatography and gel filtration chromatography of the supernatant, hepatitis B surface antigen was purified from recombinant yeast and used for assay.

### EXAMPLE 3

### Preparation of Hepatitis B Surface Antigen Conjugated to Horse Radish Peroxidase (HRP)

40 ml of the purified recombinant hepatitis B surface antigen solution prepared from the above Example 2 was concentrated to 1 ml by repeated centrifugations (3,000 rpm, 10 minutes) in Centriprep (Amicon, USA). While protein concentration was determined by commercially available BCA test kit, the concentrate was diluted with PBS to a final protein concentration of 10 mg/ml. The 10 mg/ml antigen solution was dialyzed for 1 day at 4° C. against 1 L of 0.01M sodium carbonate buffer solution, pH 9.6. Buffer solution was exchanged 3 times during the dialysis. 5 mg of HRP was quantified and dissolved in 0.5 ml of distilled water in a tube. To oxidize HRP, 100 μl of 42 mg/ml NalO₄ was added to 0.5 ml of 10 mg/ml HRP solution. After the tube was wrapped with foil, it was shaken for 30 minutes at room temperature for the oxidation reaction. To terminate the oxidation reaction, 60 μl of 1M glycerol was added to the above solution. Thereafter, the tube wrapped with foil was shaken for 1 hour at room temperature for the reaction. For a conjugation reaction, the above solution was dialyzed for 1 day at 4° C. against 1 L of 0.01M sodium carbonate buffer solution, pH 9.6. Buffer solution was exchanged 3 times during the dialysis. After the dialysis, oxidized HRP solution was transferred to a new tube, and then 250 μl of the above 10 mg/ml antigen solution was added to the new tube. The tube was wrapped with foil and shaken for 3 hours at room temperature for the reaction. During the 3-hour reaction time, 4 mg/ml NaBH₄ solution was prepared (i.e., −4 mg of NaBH₄ was weighed and dissolved in 1 ml of deionized water). To stabilize the HRP-antigen conjugate, 40.8 μl of 4 mg/ml NaBH₄ solution was added to the tube, and the tube wrapped with foil was shaken at 4° C. for 2 hours for the reaction. To separate enzyme-antigen conjugates from enzymes which were not conjugated, Sepharose CL-6B column (1 cm×45 cm, Pharmacia, Sweden) which was equilibrated with PBS one day before purification of the conjugates was used. The conjugate solution was passed through the above column using PBS as buffer. The flow rate was approximately 0.4 ml/min, and each fraction was collected for 1 min. A graph representing the protein concentration for each fraction was drawn, and the fractions corresponding to the first peak in the graph were pooled. The protein concentration of the fraction pool was determined by using commercially available BCA test kit. The sample was stored at −20° C. after adding bovine serum albumin (BSA) of final concentration of 1%.

### EXAMPLE 4

### Preparation of 96-Well Plate for Assay of Antibody against Hepatitis B Surface Antigen

To each well of a 96-well plate (Nunc International, USA) for enzyme immunoassay (EIA), each 100 μl of purified hepatitis B surface antigen solution derived from plasma from the above Example 1, was added, separately. The antigen solution was diluted with 0.1M sodium carbonate buffer solution (pH 9.5) to a final protein concentration of 0.5 μg/ml and then used. The plate was sealed with a sealing tape, and left in a cold room overnight for the antigen to bind to the surface of the well. Thereafter, the sealing tape was removed, and the antigen solution was also removed. Each 250 μl of PBS containing 1% BSA was added to each well, and then the plate was left for 2 hours at room temperature. The solution in the well was removed, and then the plate was dried by being left for 1 hour at room temperature to remove the moisture. The plate was put in a hermetic container with dehumidifying agent and stored in a refrigerator at 4° C. until further use

### EXAMPLE 5

### Assay of Antibody against Hepatitis B Surface Antigen

After plasma and recombinant antigens were obtained by using the methods in Examples 1 and 2, each plate for assay was prepared by immobilizing the plasma antigen or the recombinant antigen to a 96-well plate by using the method in Example 4, and horse radish peroxidase(HRP) was conjugated to the plasma antigen or the recombinant antigen by using the method in Example 3 for assay of antibody against HBsAg. Diagnostic experiments for detecting antibody were carried out for 4 different combinations, depending on the combination of antigen immobilized to the plate and enzyme-conjugated antigen(A group: recombinant antigen immobilized to the plate and recombinant antigen-enzyme conjugate; B group: recombinant antigen immobilized to the plate and plasma antigen-enzyme conjugate; C group: plasma antigen immobilized to the plate and recombinant antigen-enzyme conjugate; D group: plasma antigen immobilized to the plate and plasma antigen-enzyme conjugate).

Each 100 μl of antibody-negative plasma sample or antibody -positive plasma sample, was added to each well of the prepared plates. Thereafter, each 25 μl of the conjugate solution was added to each well, and then the mixture was well mixed by gently tapping the frame of the plate. The plate was transferred to a reactor at 37° C. to carry out the reaction for 60 minutes.

The wells were washed 5 times with each 300 μl of PBS containing Tween 20, and then each 100 μl of the substrate solution (100 μg/ml tetramethyl benzidine, 0.006% hydrogen peroxide, citric acid phosphate buffer solution at pH 4.5) was added to each well. After the color development in a dark place for 30 minutes, each 100 μl of the reaction-stopping solution (2N sulfuric acid) was added to each well. The absorbance was measured at 450 nm (reference wavelength, 620 nm) by using the 96 well plate reader (Molecular Devices, USA). The results were shown in Table 1 below.

TABLE 1

| Combination | A group | B group | C group | D group |
|---|---|---|---|---|
| Negative plasma | 0.129 | 0.012 | 0.019 | 0.032 |
| Positive plasma | 1.984 | 0.832 | 1.360 | 0.751 |
| Positive/negative | 15.03 | 69.33 | 74.57 | 23.46 |

From the results in the above Table 1, it was confirmed that nonspecific reaction rate was high in case that combination of same kind of HBsAgs were used for primary and secondary antigen. On the other hand, B and C groups, where combination of different kinds of HBsAgs were used for primary and secondary antigen, had low nonspecificity and high positive/negative levels.

### EXAMPLE 6

### Comparison of Sensitivities with Commercially Available Products

Comparison test of sensitivity was carried out between four different commercially available diagnostic reagents for antibody against hepatitis B surface antigen (Comparative examples 1 to 4) and the diagnostic reagent of C group according to Example 5 (the example of the present invention). In Table 2 below, diagnostic reagent of Comparative example 1 was AUSAB® EIA from Abbott Laboratories, USA; diagnostic reagent of Comparative example 2 was Enzygnost® Anti-HBs micro from Dade Behring, Germany; diagnostic reagent of Comparative example 3 was GENEDIA® Anti-HBs ELISA 3.0 from Green Cross, Korea; and diagnostic reagent of Comparative example 4 was DongA Anti-HBs ELISA from DongA Pharmaceutical Co., Korea.

The antibody used for the sentitivity test was WHO (World Health Organization) standard antibody. The absorbances were measured with 1, 2.5, 5, 10, 50 and 100 mlU/ml of WHO standards to determine the sensitivity. The results were shown in Table 2 below.

TABLE 2

| | | | | | | | 100 |
|---|---|---|---|---|---|---|---|
| Product | 0 | 1 | 2.5 | 5 | 10 | 50 | (mIU/ml) |
| Comparative | 0.003 | 0.006 | 0.014 | 0.032 | 0.079 | 0.517 | 0.867 |
| example 1 | | | | | | | |
| Comparative | 0.024 | 0.035 | 0.063 | 0.104 | 0.205 | 0.545 | 1.224 |
| example 2 | | | | | | | |
| Comparative | 0.011 | 0.017 | 0.024 | 0.045 | 0.061 | 0.282 | 0.566 |
| example 3 | | | | | | | |
| Comparative | 0.042 | 0.052 | 0.076 | 0.091 | 0.137 | 0.580 | 1.093 |
| example 4 | | | | | | | |
| Example | 0.005 | 0.043 | 0.130 | 0.252 | 0.446 | 1.717 | 2.216 |

The cut-off value of the products in general is determined by adding 0.05˜0.07 to the negative values.

As shown in the above Table 2, each diagnostic reagent of Comparative example 1, 2, 3 and 4 showed positive signal when each concentration of the antibody is more than 10 mlU/ml, 5 mlU/ml, 10 mlU/ml and 10 mlU/ml, respectively. In contrast, diagnostic reagent according to the example of the present invention, showed positive signal when concentration of the antibody is more than 2.5 mlU/ml, and therefore it had higher sensitivity than those of Comparative examples.

It should be understood that the above examples are intended to be exemplary only and not limiting the present invention. Although the present invention has been described in detail with reference to the above specific embodiments, other embodiments are possible. Therefore, it should be apparent to those skilled in the art that various modifications and changes thereof can be made without departing from the spirit and scope of the invention and that such modifications and changes be included in the scope of the following claims.

1. A diagnostic reagent for antibody against hepatitis B surface antigen using a sandwich immunoassay as a quantitative or qualitative analysis method for antibody, against hepatitis B surface antigen, characterized in that the diagnostic reagent comprises a pair of hepatitis B surface antigens which are different each other and composed of primary antigen immobilized on a solid support and secondary antigen conjugated to a marker.

2. The diagnostic reagent for antibody against hepatitis B surface antigen according to claim 1, wherein the diagnostic reagent comprises surface antigen derived from plasma and recombinant surface antigen as said pair of hepatitis B surface antigens.

3. The diagnostic reagent for antibody against hepatitis B surface antigen according to claim 2, wherein said recombinant surface antigen comprises one derived from yeast or one derived from animal cells.

4. The diagnostic reagent for antibody against hepatitis B surface antigen according to claim 1, wherein the diagnostic reagent comprises two surface antigens which are different each other by different purification methods as said pair of hepatitis B surface antigens.

5. The diagnostic reagent for antibody against hepatitis B surface antigen according to claim 1, wherein said primary antigen immobilized on the support is surface antigen derived from plasma, and said secondary antigen conjugated to the marker is recombinant surface antigen.

6. The diagnostic reagent for antibody against hepatitis B surface antigen according to claim 1, wherein said primary antigen immobilized on the support is recombinant surface antigen, and said secondary antigen conjugated to the marker is surface antigen derived from plasma.

7. The diagnostic reagent for antibody against hepatitis B surface antigen according to claim 1, wherein said marker includes an enzyme, a radioactive substance, a micro-particle, a dye or the like.

8. A diagnostic reagent for antibody against hepatitis B virus using a sandwich immunoassay as a quantitative or qualitative analysis method for antibody against hepatitis B virus, characterized in that the diagnostic reagent comprises a pair of hepatitis B surface antigens which are different each other and composed of primary antigen immobilized on a solid support and secondary antigen conjugated to a marker.

9. The diagnostic reagent for antibody against hepatitis B virus according to claim 8, wherein the diagnostic reagent comprises surface antigen derived from plasma and recombinant surface antigen as said pair of hepatitis B surface antigens.

10. The diagnostic reagent for antibody against hepatitis B virus according to claim 9, wherein said recombinant surface antigen comprises one derived from yeast or one derived from animal cells.

11. The diagnostic reagent for antibody against hepatitis B virus according to claim 8, wherein the diagnostic reagent comprises two surface antigens which are different each other by different purification methods as said pair of hepatitis B surface antigens.

12. The diagnostic reagent for antibody against hepatitis B virus according to claim 8, wherein said primary antigen immobilized on the support is surface antigen derived from plasma, and said secondary antigen conjugated to the marker is recombinant surface antigen.

13. The diagnostic reagent for antibody against hepatitis B virus according to claim 8, wherein said primary antigen immobilized on the support is recombinant surface antigen, and said secondary antigen conjugated to the marker is surface antigen derived from plasma.

14. The diagnostic reagent for antibody against hepatitis B virus according to claim 8, wherein said marker includes an enzyme, a radioactive substance, a micro-particle, a dye or the like.

15. A method of preparing a diagnostic reagent for antibody against hepatitis B surface antigen using a sandwich immunoassay as a quantitative or qualitative analysis method for antibody against hepatitis B surface antigen, characterized in that the method comprises a step of including a pair of hepatitis B surface antigens which are different each other and composed of primary antigen immobilized on a solid support and secondary antigen conjugated to a marker in the diagnostic reagent.

16. A method of preparing a diagnostic reagent for antibody against hepatitis B virus using a sandwich immunoassay as a quantitative or qualitative analysis method for antibody against hepatitis B virus, characterized in that the method comprises a step of including a pair of hepatitis B surface antigens which are different each other and composed of primary antigen immobilized on a solid support and secondary antigen conjugated to a marker in the diagnostic reagent.

17. A diagnostic method for antibody against hepatitis B surface antigen using a sandwich immunoassay as a quantitative or qualitative analysis method for antibody against hepatitis B surface antigen, characterized in that the diagnostic method utilizes a pair of hepatitis B surface antigens which are different each other and composed of primary antigen immobilized on a solid support and secondary antigen conjugated to a marker.

18. A diagnostic method for antibody against hepatitis B virus using a sandwich immunoassay as a quantitative or qualitative analysis method for antibody against hepatitis B virus, characterized in that the diagnostic method utilizes a pair of hepatitis B surface antigens which are different each other and composed of primary antigen immobilized on a solid support and secondary antigen conjugated to a marker.
